(19) United States
(12) Patent Application Publication
(10) Pub. No.: US 2008/0091472 A1
(43) Pub. Date: Apr. 17, 2008

(54) TREATMENT MONITORING TOOL

(76) Inventor: Steven Hoppe, Edmonds, WA (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(21) Appl. No.: 11/870,785

(22) Filed: Oct. 11, 2007

Related U.S. Application Data
(60) Provisional application No. 60/829,146, filed on Oct. 11, 2006.
(60) Provisional application No. 60/892,050, filed on Feb. 28, 2007.

(51) Int. Cl. A61B 5/00; G06F 7/00

(52) U.S. Cl. 705/3; 707/104.1

(57) ABSTRACT

A tool for monitoring a course of treatment for a patient, including standards of care making up the course of treatment. The tool permits tracking of multiple patients, their diagnoses, corresponding treatment regimens, and status of those regimens. Once a diagnosis is selected by a physician, the embodiment may list the various steps and/or actions undertaken in a recommended course of treatment for that diagnosis. Such steps/actions can include tests to be performed on the patient, drugs or prescriptions to be prescribed to the patient, physical activity to be undertaken by the patient, assessments to be made by a practitioner, environmental factors to be applied to the patient and so forth. The tool also lists particular standards of care for a given diagnosis, as well as which standards of care are incomplete. The tool may also present a patient's location.

### BACKGROUND OF THE INVENTION

1. Technical Field

An embodiment of the present invention relates generally to a tool for monitoring a standard of treatment for a patient, and more particularly to a computer-implemented software application for monitoring a standard of treatment based on one or more diagnoses.

2. Background Art

As man's understanding of medicine and disease has progressed, treatments for various conditions have become both more effective and, in many cases, more complex. Even relatively simple procedures now may have multiple steps including physical operations, prescription of medicines, running of tests and so forth. Diagnosis of a single condition in a patient may require the application of multiple standards of care to meet, treat, or cure the diagnosis. The act of applying or following each of these standards of care is referred to herein as “treatment” or a “course of treatment.” This increased complexity in accepted standards of care may make caring for a patient more difficult and may lead to health care providers occasionally forgetting or neglecting a standard of care. Although certain standards of care may be omitted in some cases, many times each and every standard of care is necessary or suggested for a particular patient or diagnosis.

Further, hospitals, hospices, nursing homes and other places caring for many patients at once have an even greater difficulty compounded by the fact that they have multiple patients. Each patient may have multiple diagnoses, each of which may have multiple standards of care. Thus, it is conceivable that a single hospital might need to track and apply thousands of standards of care for hundreds of patients. In such an environment, it is particularly difficult to determine whether or not each standard of care has been ordered and/or completed for every patient and every diagnosis.

Accordingly, there is a need in the art for an improved tool and method for monitoring a patient, the patient's diagnosis and treatment.

### SUMMARY OF THE INVENTION

Generally, one embodiment of the present invention takes the form of a tool for monitoring a course of treatment for a patient, including standards of care. The embodiment may be implemented as a series of computer-executable instructions (i.e., software) or dedicated computer architecture particularly designed to carry out the operations described herein (i.e., hardware). The embodiment may operate on any computing device, including, but not limited to: a personal computer; a personal digital assistant or other handheld computing device; minicomputer; distributed computing architecture; server; mobile computing device (including a mobile telephone); and so forth.

The exemplary embodiment permits tracking of multiple patients, their diagnoses, corresponding treatment regimens, and status of those regimens. For example, once a diagnosis is selected by a physician, the embodiment may list the various steps and/or actions undertaken in a recommended course of treatment for that diagnosis. Such steps/actions may include, but are not limited to, tests to be performed on the patient, drugs or prescriptions to be prescribed to the patient, physical activity to be undertaken by the patient, assessments to be made by a practitioner, environmental factors to be applied to the patient and so forth. These various operations and/or factors are collectively referred to as a “course of treatment” and individually as “standards of care.”

One exemplary embodiment of the present invention takes the form of a standards monitoring tool, including a first database storing a first plurality of records, each of the first plurality of records corresponding to a diagnosis, a second database storing a second plurality of records, each of the second plurality of records corresponding to a standard of care, each standard of care further corresponding to at least one diagnosis, and a third database storing a third plurality of records, each of the third plurality of records corresponding to a patient, wherein each patient is associated with at least one diagnosis and at least one standard of care; and selecting one of the third plurality of records also selects at least one of the first plurality of records and at least one of the second plurality of records. It should be noted that any or all of the first, second and third databases may be the same database.

Yet another embodiment of the present invention takes the form of a computer-implemented method for monitoring a standard of care for at least a first patient, including the operations of receiving a diagnosis for the first patient, retrieving at least one standard of care from a first database, the at least one standard of care corresponding to the diagnosis, and providing an indicator associated with the standard of care, the indicator indicating at least if the standard of care has been met. The embodiment may also include the operations of storing the diagnosis as a record in a second database and storing a patient identifier associated with the first patient in a third database, wherein retrieving the patient identifier likewise retrieves the diagnosis and the at least one standard of care.

Still another embodiment of the present invention takes the form of a method for generating a report, including the operations of retrieving, from a first database, a patient record, retrieving, from a second database, a diagnosis associated with the patient record, retrieving, from a third database, a plurality of standards of care associated with the diagnosis (each of the standards of care comprising a first datum indicating if the standard of care has been initiated, a second datum indicating if the standard of care has been finished and a third datum indicating if the standard of care is excepted), determining through each of the plurality of third data if each of the plurality of standards of care is an excepted standard of care, and compiling each excepted standard of care into the report.

Additional embodiments and capabilities of the present invention will be apparent to those of ordinary skill in the art upon reading this disclosure in its entirety.

### BRIEF DESCRIPTION OF THE FIGURES

FIG. 1 depicts an exemplary embodiment of the invention.

FIG. 2 depicts a census screen in accordance with the exemplary embodiment of the invention.

FIG. 3 depicts a care standards screen in accordance with the exemplary embodiment of the invention.

FIG. 4 depicts an exception screen in accordance with the exemplary embodiment of the invention.

### DETAILED DESCRIPTION OF THE INVENTION

I. Overview and Introduction

Generally, one embodiment of the present invention takes the form of a tool for monitoring a course of treatment for a patient. The embodiment may be implemented as a series of computer-executable instructions (i.e., software) or dedicated computer architecture particularly designed to carry out the operations described herein (i.e., hardware). The embodiment may operate on any computing device, including, but not limited to: a personal computer; a personal digital assistant or other handheld computing device; minicomputer; distributed computing architecture; server; mobile computing device (including a mobile telephone); and so forth.

The exemplary embodiment permits tracking of multiple patients, their diagnoses, corresponding treatment regimens, and statuses of those regimens. For example, once a diagnosis is selected by a physician, the embodiment may list the various steps and/or actions undertaken in a recommended course of treatment for that diagnosis. Such steps/actions may include, but are not limited to, tests to be performed on the patient, drugs or prescriptions to be prescribed to the patient, physical activity to be undertaken by the patient, assessments to be made by a practitioner, environmental factors to be applied to the patient and so forth. These various operations and/or factors are collectively referred to as a “course of treatment.”

The course of treatment specified by the exemplary embodiment corresponds with the best practices of the medical profession and/or specialized treatments designed by a user. The embodiment may, however, provide alternative courses of treatment in addition to a standard course of treatment. Further, the embodiment may be configured to provide custom courses of treatment instead of standard courses of treatment by changing the database, as described below.

The embodiment may not only track patients and their courses of treatment, but also any exceptions to the courses of treatment. Further, the embodiment may track when a patient is discharged from care. Each patient, the corresponding course of treatment, and all other patient information is typically stored as a database record accessible by the embodiment. As the patient's status (for example, under treatment or discharged), course of treatment, or other patient-related information changes, the embodiment may update the corresponding database record.

Having generally provided an overview of an exemplary embodiment, the operation of various embodiments of the present invention will now be discussed.

II. General Operation of an Exemplary Embodiment

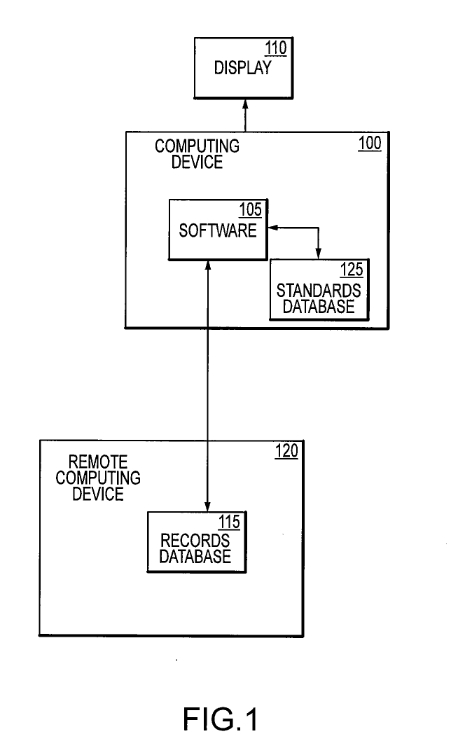

FIG. 1 generally depicts one exemplary embodiment of the present invention. A computing device 100 may be accessed by a user. Software 105 (or, in some embodiments, properly configured hardware) may run on the computing device 100. The user may operate the computing device 100 to activate the software 105 in order to perform the various functions described herein. The software 105 may present patient records, diagnoses, associated care standards and so forth on a display 110 for the user's review.

The software 105 generally may access a records database 115. The records database 115 typically stores one or more records, each of which contains information for a particular patient. The content of each record is discussed in more detail below, but broadly contains all patient data and/or information discussed herein with the exception of the standards of care.

The records database 115 may be stored or located on a remote computing device 120 and accessed across a network by the software 105. It should be noted that the remote computing device 120 may be replaced by a remote storage element (such as a remote magnetic storage device or optical storage device) in certain embodiments. Alternatively, the records database may be stored on the same computing device 100 as the software 105.

A standards database 125 may contain the various standards of care for each diagnosis. Thus, when a user enters a particular diagnosis for a patient, the standards of care corresponding to that diagnosis may be retrieved from the standards database 125. Similarly, when a patient record is accessed from the records database 115 by the software 105, the standards of care for each diagnosis indicated in the patient record may be retrieved from the standards database 125. In some embodiments, the standards database 125 and records database 115 may be combined into a single database.

The standards database 125 is shown in FIG. 1 as resident on the same computing device 100 as the software 105. In alternative embodiments, it may be located on a remote computing device 120 or remote storage element.

If a user updates any information in a patient record via the software 105, the corresponding record in the records database 115 may likewise be updated.

III. Census Screen

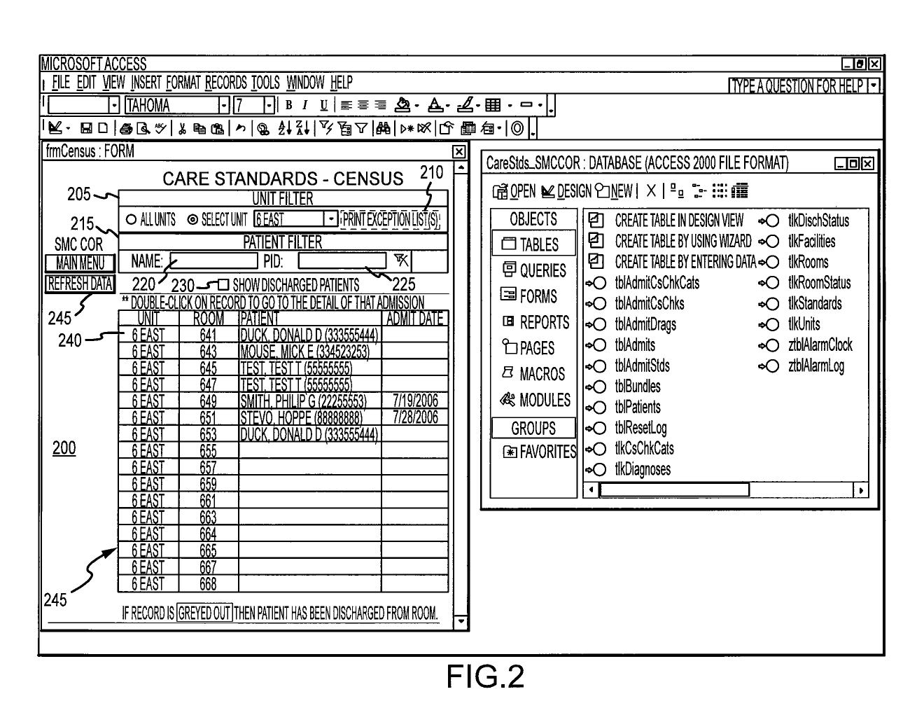

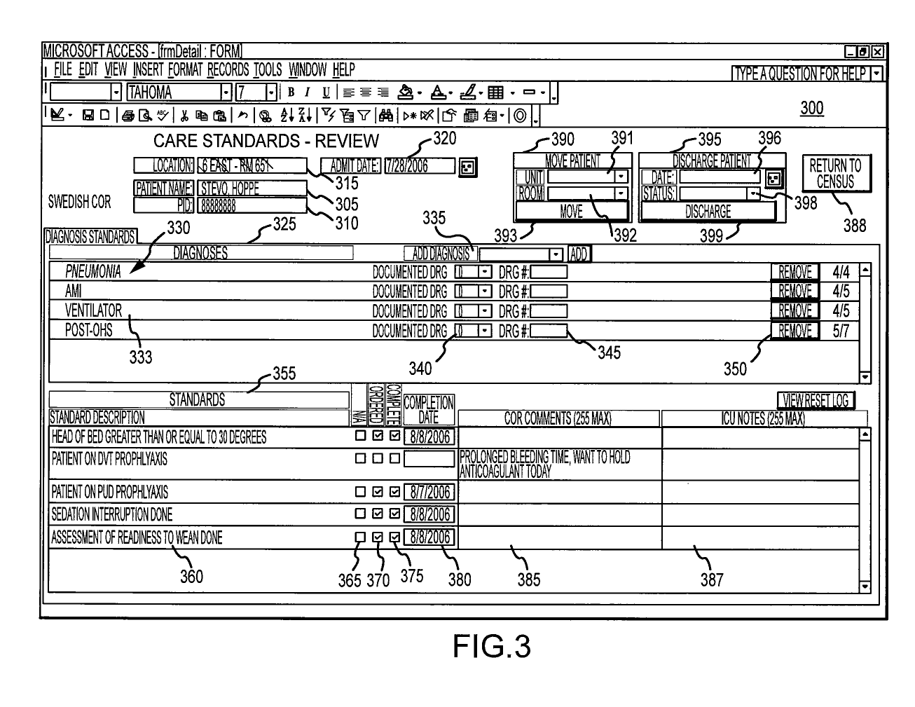

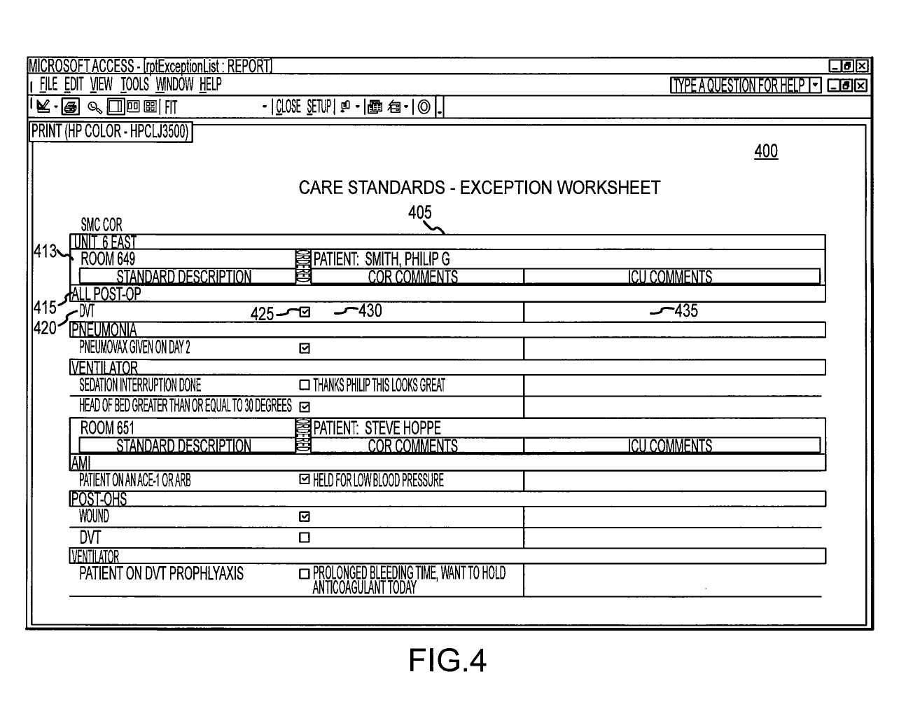

FIG. 2 depicts a census screen 200 of an exemplary embodiment of the present invention. In the present embodiment, the census screen 200 permits a user to select a patient for detailed review through a care standards screen as described below with respect to FIG. 3, and access an exception screen 400 as detailed below with respect to FIG. 4. Patients that may be selected by a user are generally displayed in a patient list 245.

The patient list may be searched in a variety of ways. First, a user may employ the unit filter 205 to filter the patient list 245 in order to show only patients in a particular unit. “Unit,” as used here, generally refers to a unit of a hospital or hospital system, but may refer to any geographic area such as a ward, wing, or even town or hospital. The user may either select a particular unit through a drop-down menu or may display patients for all units.

In some embodiments, the user may also filter the patient list 245 by using the patient filter 215. The patient filter permits a user to specify a patient's name or record identifier in the name field 220 and identifier field 225, respectively. A patient's record identifier is typically an alphanumeric string and may correspond to a hospital record number, insurance number, patient record number, or may be unique. In either case, only patients matching data inputted into either or both of the name field 220 and identifier field 225 will be shown in the patient list 245. Thus, if the user types “John” into the name field 220, only patients having “John” in any portion of their names will be shown. In this example, the patient list 245 would show both “John Smith” and “Steve Johnson,” as “John” is the first name of one and part of the last name of the other.

A user may double-click or otherwise select a row 240 of the patient list 245 to choose that particular patient. When a patient is selected, the embodiment displays the care standards screen of FIG. 3, discussed in more detail below.

The user may likewise generate an exception list 400, described below with respect to FIG. 4, by selecting the exception list button 210. Each exception list 400 is generated for a unit specified in the unit filter 205 or, if no unit is specified, for all patients in the records database 115.

IV. Care Standards Screen

FIG. 3 depicts an exemplary care standards screen 300 in accordance with the exemplary embodiment. In this screen 300, a user may review the various diagnoses and associated standards of care for a given patient. The user may see the patient's name in the patient field 305 and the patient's record identifier in the identification field 310. Records in the database may be indexed by medical record instead of patient name to avoid data errors that may occur when two patients have identical names. Each record identifier may be any unique alphanumeric string and typically corresponds in format to the format employed by a hospital, nursing home, clinic, hospice, outpatient center or other care facility employing or operating the exemplary embodiment. As an alternative, the record field 215 may display a patient's insurance policy number or other identifying insurance information instead of a medical record number.

When the record identifier (or, in some embodiments, the patient's name) is entered into the corresponding field of the census screen 200 and the care standards screen 300 is accessed, the embodiment will retrieve the database record for the associated patient and display the associated information in the care standards screen 300. For example, the record may be used by the embodiment to populate various fields, such as the patient field 305, location field 315, admit date field 320, diagnoses field 325, standards field 355, and so on. Changing the value of any field so populated will update the related database record to reflect the change.

The location field 315 generally displays the present location of the patient. The location may be chosen from among a drop-down menu or may be entered by a user. Typically, these are rooms, wings or wards within a hospital system, but may instead correspond to various hospitals (optionally with sublocations such as rooms) within a multi-hospital system. Additional locations, such as the patient's home, a hospice, outpatient center, nursing home and so forth may also be selectable options for the location field 210. A user may select the patient's location from the drop-down menu.

The admit date field 320 shows the date of the patient's admission to the care facility.

The diagnosis area 325 generally lists the various diagnoses assigned to a patient. As a doctor or other care provider diagnoses a patient, he or she may enter into the patient's record the determined diagnosis. A new diagnosis may be entered for a patient through the add diagnosis field 335. In the present embodiment, the add diagnosis field 335 permits a user to select a new diagnosis from a drop-down menu. Alternative embodiments may permit a user to type the diagnosis directly into the add diagnosis field or into the diagnosis list 330. Adding a diagnosis to the diagnosis area 325, via the add diagnosis field 335, may prompt the embodiment to retrieve the standards of care associated with the added diagnosis from the standards database 325. Alternatively, the standards of care for a given diagnosis may be retrieved from the standards database 325 only when a particular diagnosis row 330 is selected by the user, as described below.

Each diagnosis for a patient is listed in the diagnosis list 330. The diagnosis list 330 is made of a series of diagnosis rows 333, each of which shows a separate diagnosis. In an exemplary embodiment, each diagnosis row has a corresponding DRG field 340 and DRG number field 345. (The DRG field, DRG number field, and associated data may be omitted in alternative embodiments, in which case no such data is stored in the database.) The DRG field 340 and DRG number field 345 permit classification of the diagnosis, and thus the patient, into a particular diagnosis-related group. There are approximately 500 diagnosis-related groups in use by hospitals. Diagnosis-related groups are broken down according to subject. At the time of filing, approximately 25 subject areas exist and 490 diagnosis-related groups are split between these subject areas. For example, subject area 22 is “burns;” diagnosis-related groups 456-460 are assigned to this subject area. The diagnosis-related groups and their functions are well known to doctors and other health care providers. A user may select a particular subject area in the DRG field 340. Likewise, the user may enter the number of the relevant diagnosis-related group into the DRG number field 345. A diagnosis may be a physical condition of the patient, an illness or health issue experienced by the patient, a medication the patient should take, or an apparatus to which the patient should be connected.

A particular diagnosis may be removed from the care standards screen 300 by clicking or otherwise selecting the remove button 350. Selecting the remove button 350 will also delete the diagnosis from the patient record stored in the records database 115.

Each diagnosis displayed in the diagnosis field 325 has a related set of standards of care. Selecting a diagnosis row 330 causes the standards of care for the corresponding diagnosis to be retrieved from the standards database 125 and displayed in the standards area 355. The standards area 355 may have multiple standards rows 360, each of which is a separate standard of care for the selected diagnosis. Generally, the standards of care for a given diagnosis set forth the treatment necessary for the diagnosis. By displaying all the standards of care for a selected diagnosis, the embodiment enables the user to quickly and easily see if the standards have been completed or initiated. This, in turn, may permit quicker and more effective care for a patient.

When a standard of care is ordered or initiated, the user may check the ordered box 370 to indicate the standard of care has been ordered/initiated. Upon completion of the standard of care, the user may check the complete box 375. In some embodiments, checking the complete box 375 automatically enters the date on which the box was checked in to the completion date field 380. In alternative embodiments, the user may be required to manually input the completion date for the standard of care. The user may generally override any automatic entry in the completion date field 380 by specifying an input.

In the event the user determines a particular standard of care is not necessary for a patient, he may check or select the corresponding N/A box 365. Selecting the N/A box removes the standard of care from the Exception screen, discussed below in Section VI. It also removes the standard of care from any exception report generated, as discuss below in Section VII.

Each standard of care row 360 includes two comment fields, specifically a first comment field 385 and a second comment field 387. A user may enter comments regarding the associated standard of care in either comment field. For example, the user may enter a comment indicating why a particular standard of case was deemed not applicable or not ordered. Although the two comment fields 385, 387 are separate, any user may enter a comment in either field. Alternative embodiments may restrict a user from entering a comment in one or the other field based on the user's access privileges.

As an example of the interplay between the diagnosis area 325 and the standards area 355, in FIG. 3 the ventilator diagnosis row 330 is selected. Five standards of care are listed in the standards area 355, each on a separate standards row 360. Each standard is an activity, physical condition, test, or medication that should be performed or given to the patient; each standard is also associated with the ventilator diagnosis. Continuing the example, the first standard of care listed in the standards area for the ventilator diagnosis is “Head of bed greater than or equal to 30 degrees.” This standard of care instructs the care provider to adjust the head of the patient's bed to angle upward at least 30 degrees. The example of FIG. 3 further shows that both the ordered box 307 and complete box 375 have been checked, indicating that the standard of care has been both ordered and completed. Further, the completion field 380 indicates the standard of care was completed on Aug. 8, 2006.

Certain standards of care may not be complete, in which case the complete box 375 is not checked. As discussed in more detail below, incomplete standards of care may show on an exception screen 400.

The care standards screen 300 also permits a user to move or discharge a patient via the move patient area 390 and discharge patient area 395, respectively. The move patient area 390 includes a unit field 391, a room field 392 and a move button 393. A user may select a particular unit (which may be a hospital unit, ward, wing, etc.) from a drop-down menu in the unit field 391. The embodiment may populate the room field 392 with the rooms in the selected unit, which may be retrieved from an appropriate listing or database. The user may then select a room from the room field 392, thus specifying both the unit and room to which the patient is to be (or has been) moved. Clicking the move button 393 updates the patient record to reflect the new unit and room specified in the corresponding fields 391, 392.

Similarly, a user may discharge a patient from care from the care standards screen 300. Discharging a patient flags the patient record so that it is not retrieved during operation of the embodiment. Alternatively, in certain embodiments the record may be retrieved and the patient shown as discharged in screens such as the census screen 200 and/or standards screen 300. The discharge area 395 includes a date field 396, stats field 398 and discharge button 399. The date field displays the date on which a patient is discharged, if the patient has been discharged. A user may also enter a discharge date into this field. (In alternative embodiments where the patient record of a discharged patient may not be accessed, no date is initially displayed in the discharge area; a date may only be entered by a user.) The status field 398 allows a user to select from amongst a list of statuses for the discharged patient. It should be noted that the status field is entirely optional and may be omitted in many embodiments of the present invention. The discharge statuses may reflect the condition of the patient upon discharge. In alternative embodiments, the user may enter a status into the status field 398 instead of selecting from a drop-down list. The user may discharge the patient by clicking the discharge button 399, which updates the record as described above (i.e., by appropriately flagging the record or deleting the record). In some embodiments, clicking the discharge button 399 may add the date on which the button was clicked to the date field 396 and/or a default status to the status field 398. In other embodiments, such information may be added only to an otherwise blank field.

Selecting the return button 388 will return the user from the standards screen 300 to the census screen 200.

V. Creating New Patient Records

In the present embodiment, a new patient record may be created directly from the census screen 200 of FIG. 2. For example, a patient name may be entered into the name field 220 when it is blank in order to initiate creation of a new record. Likewise, a patient identifier may be entered into a blank identifier field 225 to initiate creation of a new record in an embodiment having filtering functionality, a button, box, or other selectable item may be provided to differentiate creation of a new record from filtering the database records according to information in the name field 220 or identifier field 225. As yet another option, a button, box, or other selectable item may be present on the census screen 200. When such a selectable item is chosen or clicked, the embodiment may create a new record that may be populated by the user.

In certain alternative embodiments, the user may access a blank care standards screen generally similar to the care standards screen 300 of FIG. 3. By entering data into the blank care standards screen, the user may create a new record in the records database 115. The user may thus specify such information as a new patient's name, record identifier, admission date, and any diagnoses, all of which are added as a record in the records database.

In yet other embodiments, creation of a new record may be initiated from a differently-configured screen having fields for entry of appropriate information, such as a patient name and/or record identifier. Other information may be entered from the care standards screen 300 and/or exception screen 400 as necessary.

VI. Exception Screen

FIG. 4 generally depicts an exemplary exception screen 400, in accordance with the present exemplary embodiment. The exception screen 400 of the exemplary embodiment includes a unit field 410. The unit field specifies the unit for all patients shown on the exception screen 400 and matches the unit selected in the unit filter 205 of the census screen 200. For each patient in the unit, a separate list of exceptions is shown as discussed in more detail below. Patients shown on the exception screen 400 are typically listed in alphabetical order by last name, although this may vary in alternative embodiments.

For each patient, a list of all diagnoses and all standards of care corresponding to a diagnosis for a patient that have not been completed (referred to as “exceptions”) is shown. The exceptions are shown beneath each patient name on a unique exception row 420; each exception row 420 is also shown beneath a diagnosis row 415 listing the diagnosis to which the exception is linked. The exceptions are also listed on the care standards screen 300 as separate standards of care, each on its own standards row 360, insofar as each exception is a also a standard of care associated with a diagnosis listed in the diagnoses area 325.

Generally, the embodiment generates the exception screen 400 in order to display to a user all standards of care for a patient that are not yet complete. The user may then, relatively quickly and easily, determine which standards of care still are to be completed (or, at least, are still listed to be completed). As shown in FIG. 4, each diagnosis having an exception is listed on a separate diagnosis line 415. Every exception for a given diagnosis is listed beneath the corresponding diagnosis line; each such exception is listed on an exception line 420. Multiple exception lines 420 may correspond to a single diagnosis line 415.

Each exception line 425 lists the standard of care that has not yet been completed. Further, each exception line 420 includes an exception ordered checkbox 425, similar to the ordered box 370 on the care standards screen 300. As with the ordered box 370 of the care standards screen 300, the exception ordered checkbox 425 indicates whether or not the particular exception has been ordered. Checking or unchecking (i.e., updating) the exception ordered checkbox 425 changes the status of the ordered box 370 to match. Similarly, the status of the exception ordered checkbox 425 always matches the ordered box 370 status, so too are changes to the ordered box are reflected in the exception ordered checkbox. In some embodiments, checking the ordered box 425 removes the associated standard of care from the exception screen 400.

Each exception line 425 also includes a first and second exception comment field 430, 435. These exception comment fields 430, 435 match the first and second comment fields 385, 387 of the care standards menu 300. Any comment in the first comment field 385 of the care standards menu 300 is shown in the first exception comment field 430. Likewise, any comment in the second comment field 387 of the care standards menu 300 is shown in the second exception comment field 430. Changes to one of the exception comment fields are reflected in the comment fields of the care standards menu, and vice versa.

The exception screen 400 also displays a patient's name in the patient name field 405. and a patient's location in the patient location field 413. The location shown in the patient location field 413 typically matches the location shown in the location field 315 of the care standards menu 300. Changes to the patient's location entered via the movie patient area 390 are reflected in the patient location field 413.

VII. Reports

Generally, the exemplary embodiment permits a user to assess current and past patient care in order to implement identified practice standards related to various diagnoses or treatment modalities, both for individual patients and groups of patients. One assessment tool available to a user of an embodiment is a report that may be generated by the embodiment.

As a first example, an exemplary embodiment may produce an exception list or report. The exception list may contain all exceptions for a given patient. Certain embodiments may also generate a report listing all exceptions associated with a given diagnosis, regardless of to which patient each listed exception corresponds. Additionally, still other embodiments may generate a report listing all exceptions for a given treatment modality, regardless of the patients or diagnoses associated with the modality. Still other embodiments may generate a report listing all exceptions associated with a particular group of patients. Yet other embodiments may generate a report showing all diagnoses associated with a patient or group of patients. Still further embodiments may generate reports showing all completed or initiated standards of care associated with a patient or group of patients. A “group of patients” may be all patients selected by a user, all patients in a common geographic area, all patients sharing a common diagnosis, or any other grouping of patients. In short, the various data discussed herein may be grouped in a variety of fashions and reported accordingly.

VIII. Conclusion

It should be understood that the formatting of the information gathered, tracked and/or displayed by the exemplary embodiment described herein may vary in alternative embodiments. For example, the various standards of care or exceptions may be listed in different orders, formatted differently, and so forth. As yet another example, an alternative embodiment may lack any standards rows 360 and/or exception lines 420, instead displaying such information in a separate window when a diagnosis row 330 or diagnosis line 415 is selected by the user. Additionally, various fields, areas, and other information may be omitted from the screens 200, 300, 400 described herein, or additional fields, areas, and/or information may be added to the screens. Accordingly, the configuration of the various screens 200, 300, 400 should be understood to be exemplary and not limiting. In a similar manner, any field permitting user input and described herein may be replaced by a drop-down box and vice versa. Thus, the proper scope of the invention is defined by the following claims.

What is claimed is:

1. A standards monitoring tool, comprising:
a first database storing a first plurality of records, each of the first plurality of records corresponding to a diagnosis;
a second database storing a second plurality of records, each of the second plurality of records corresponding to a standard of care, each standard of care further corresponding to at least one diagnosis;
a third database storing a third plurality of records, each of the third plurality of records corresponding to a patient; wherein
each patient is associated with at least one diagnosis and at least one standard of care; and
selecting one of the third plurality of records also selects at least one of the first plurality of records and at least one of the second plurality of records.

2. The standards monitoring tool of claim 1, wherein the first and second databases are a single database.

3. The standards monitoring tool of claim 2, further comprising:
a first module for depicting a selected patient, at least one diagnosis associated with the selected patient and at least one standard of care associated with the at least one diagnosis; and
a second module for depicting at least one excepted standard of care associate with the at least one diagnosis.

4. The standards monitoring tool of claim 3, wherein the at least one standard of care and at least one excepted standard of care are the same.

5. The standards monitoring tool of claim 3, further comprising:
a module for generating an exception list, the exception list listing all excepted standards of care associated with the patient.

6. The standards monitoring tool of claim 5, wherein the tool is implemented as a computing device.

7. The standards monitoring tool of claim 5, wherein the tool is implemented as a computer-readable program.

8. A computer-implemented method for monitoring a standard of care for at least a first patient, comprising:
receiving a diagnosis for the first patient;
retrieving at least one standard of care from a first database, the at least one standard of care corresponding to the diagnosis; and
providing an indicator associated with the standard of care, the indicator indicating at least if the standard of care has been met.

9. The computer-implemented method of claim 8, further comprising:
storing the diagnosis as a record in a second database; and
storing a patient identifier associated with the first patient in a third database; wherein
retrieving the patient identifier likewise retrieves the diagnosis and the at least one standard of care.

10. The computer-implemented method of claim 9, wherein at least two of the first database, second database, and third database are the same.

11. The computer-implemented method of claim 9, further comprising:
receiving a datum indicating that the at least one standard of care is an exception;
receiving a request to generate an exception list;
in response to receiving the request to generate the exception list, generating the exception list, wherein the exception list comprises the exception.

12. The computer-implemented method of claim 11, further comprising:
storing the exception list.

13. The computer-implemented method of claim 11, further comprising:
receiving a request to generate a compliance list; and
in response to receiving the request to generate the compliance list, generating a compliance list including data detailing a number of exceptions associated with one of the group comprising: the diagnosis; a treatment modality; the first patient; and a group of patients.

14. The computer-implemented method of claim 13, wherein the diagnosis is associated with a second patient in addition to the first patient.

15. The computer-implemented method of claim 9, further comprising:
receiving the patient identifier;
in response to receiving the patient identifier, displaying each of the patient identifier, the diagnosis and the at least one standard of care.

16. The computer-implemented method of claim 9, further comprising:
receiving a request to display an exception screen; and
in response to receiving the request to display the exception screen, displaying at least one standard of care associated with the patient that has not been flagged as complete.

17. The computer-implemented method of claim 9, further comprising:
receiving a request to display an exception screen; and
in response to receiving the request to display the exception screen, displaying at least one standard of care associated with the patient that has been flagged as inapplicable to the patient.

18. A method for generating a report, comprising:
retrieving, from a first database, a patient record;
retrieving, from a second database, a diagnosis associated with the patient record;
retrieving, from a third database, a plurality of standards of care associated with the diagnosis, each of the standards of care comprising:
a first datum indicating if the standard of care has been initiated;
a second datum indicating if the standard of care has been finished; and
a third datum indicating if the standard of care is excepted;
determining through each of the plurality of third data if each of the plurality of standards of care is an excepted standard of care; and
compiling each excepted standard of care into the report.

19. The method of claim 18, further comprising displaying the report on a computer screen.

20. The method of claim 18, further comprising displaying the report on paper.
